(19) United States
(12) Patent Application Publication
(10) Pub. No.: US 2021/0186824 A1
(43) Pub. Date: Jun. 24, 2021

(54) MATERIAL TO FILL DENTAL SPACES

(71) Applicant: SONENDO, INC., Laguna Hills, CA (US)

(72) Inventors: Zaza Gomurashvili, Tustin, CA (US); David Tobia, Laguna Hills, CA (US); Mehrzad Khakpour, Laguna Hills, CA (US); Bjarne Bergheim, Laguna Hills, CA (US)

(21) Appl. No.: 17/112,809

(22) Filed: Dec. 4, 2020

Related U.S. Application Data
(63) Continuation of application No. PCT/US2019/035884, filed on Jun. 6, 2019.
(60) Provisional application No. 62/681,832, filed on Jun. 7, 2018.

(51) Int. Cl. A61K 6/898; A61C 5/64; A61C 5/68; A61K 6/54; A61K 6/836; A61K 6/802; A61K 6/84; A61K 6/17; A61K 6/842

(52) U.S. Cl. CPC A61K 6/898; A61C 5/64; A61C 5/68; A61K 6/54; A61K 6/842; A61K 6/802; A61K 6/84; A61K 6/17; A61K 6/836

(57) ABSTRACT

A curable mixture and method of using the mixture are disclosed. In some embodiments, the mixture comprises an alginate polymer, and comprises properties suitable for use as a tooth filling after curing.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6. This application is a continuation of PCT International Application No. PCT/US2019/035884, filed Jun. 6, 2019, entitled “MATERIAL TO FILL DENTAL SPACES”, which claims the benefit of U.S. Provisional Patent Application No. 62/681,832, filed Jun. 7, 2018, entitled “BIOCOMPATIBLE OBTURATION MATERIAL”, the entirety of which are hereby incorporated by reference.

### BACKGROUND

In conventional endodontic procedures, an opening is drilled through the crown of a diseased tooth, and endodontic files are inserted into the root canal system to open the canal spaces and remove organic material therein. The root canal is then filled with solid matter such as gutta percha and an obturation material, and the tooth is restored. However, this procedure will not remove all organic material from the canal spaces, which can lead to post-procedure complications such as infection. In addition, motion of the endodontic file may force organic material through an apical opening into periapical tissues. In some cases, the end of the endodontic file itself may pass through the apical opening. Such events may result in trauma to the soft tissue near the apical opening and lead to post-procedure complications.

Current treatment techniques for tooth decay (caries) generally include mechanical removal of the caries and diseased tissue (e.g., using dental burs, excavators, etc.), which will expose healthy dentin. However, the bur (or other mechanical instrument) may not differentiate between diseased and healthy dentin, and other instruments such as excavators and explorers may not be able to accurately determine the extent to which tooth removal should continue. This may result in either incomplete removal of caries or overly-aggressive removal of healthy dentin, which may in turn reduce the longevity of the tooth. The removed portions of the tooth can then be filled with solid matter such as composite, resin, gold, porcelain, etc., and the tooth can be restored. However, this procedure may not remove all decayed material from the tooth, which combined with inadequate penetration of the restorative material can result in bacterial leakage and subsequently post-procedure complications such as infection or recurrent caries. In part to minimize the risk of reinfection, endodontic material placement typically requires the use of a gutta percha point to encourage penetration of the obturation material into lateral canals and isthmi. In addition, the use of a dental drill and anesthetics may be uncomfortable for the patient. Various filling spaces within or adjacent to a tooth can benefit from improvements in dental treatment techniques. Examples of such filling spaces include but are not limited to root canals, cavities resulting from the removal of caries, other openings such as cracks and gaps, and/or missing portions of teeth (e.g., resulting from fracture and/or wear). Accordingly, it can be advantageous to provide improved compositions, methods and apparatus for treating dental decay.

More recently, dental apparatuses have been developed that can deliver a curable mixture to a treatment region without the necessity of an obturation point. (See U.S. Pat. No. 9,877,801, the entire contents of which are incorporated herein by reference for all purposes). Various formulations are known that can be used as curable mixtures. However, the compatibility of current materials with the new technology is less than desired. Thus, the need for more advanced obturation materials is needed.

### SUMMARY

This disclosure is directed to a polymerizable restorative composition that is either a one-part or multi-part system and serves to replace a decayed or infected tooth structure after removal of the decay. The polymerizable restorative composition includes a curable mixture of ingredients that comprises (a) a polymerizable monomer (such as a polymerizable polysaccharide), (b) a cross-linking or activating agent (such as a divalent cation source), (c) a filler material, and (d) a carrier liquid. The polymerizable restorative composition is preferably biocompatible and has adequate physical properties after curing to function as a replacement for the decayed tooth structure.

Some embodiments described herein generally relate to a curable mixture with properties that facilitate delivery of the polymerizable restorative composition to the treatment region. The treatment region can be inside or outside the tooth. It can be the root canal space, a carious lesion, tooth abrasion, a periodontal pocket, and/or other bone structure. The curable mixture can be radiopaque so that the placement can be verified using an x-ray or cone beam imaging. The treatment region can have an initial opening and a secondary or more openings. It can be a single space or a plurality of spaces in the tooth. The cross-sectional dimensions of the spaces within the treatment region can vary from nanometers (e.g. in case of enamel caries) to millimeters (e.g. in case of dentin caries or root canal system). In some embodiments, the curable mixture is a curable mixture of ingredients selected to provide the resulting cured mixture with dimensional stability after cure. In some embodiments the cured mixture is resorbable (e.g., a resorbable alginate) with a predetermined resorption time. The cured mixture may promote soft and hard tissue regeneration. In some embodiments the cured mixture can be permanent with little to no resorption over time.

In some embodiments, the polymerizable restorative composition comprises a two-part curable mixture that comprises at least the following ingredients in the form of a part 1 and a part 2 as follows: part 1: (a) a polymerizable polysaccharide, (b) a divalent cation source, and (c) a filler material; and part 2: (d) a carrier liquid. In various embodiments the two-part curable mixture is curable by combining part 1 with a coherent collimated jet that comprises a part 2 to form a combined curable mixture that comprises the ingredients (a), (b), (c) and (d). In various embodiments, one or both of part 1 and part 2 is degassed. The ingredients (a), (b), (c) and (d) can be selected to provide the combined curable mixture with a viscosity that facilitates delivery of the combined curable mixture into a space in a tooth to form a cured mixture. The ingredients (a), (b), (c) and (d) can also be selected to provide the cured mixture with dimensional stability within the space in the tooth.

In some embodiments the curable mixture is substantially anhydrous. Some embodiments described herein generally relate to a method of restoring a tooth, comprising identifying a tooth having a cavity or abrasion in need of filling, positioning a substantially anhydrous curable mixture within the cavity, and exposing the substantially anhydrous curable mixture within the cavity to water for a period of time effective to cure the mixture. The uncured or partially cured curable mixture can be an obturation material mixture and may be simply referred to herein as a “mixture” or “curable mixture”.

Some embodiments described herein generally relate to a method of filling a root canal, comprising identifying a tooth having a root canal in need of filling, positioning a substantially anhydrous curable mixture within the root canal, and exposing the substantially anhydrous curable mixture within the root canal to water for a period of time effective to cure the mixture.

Some embodiments described herein generally relate to a method of filling a tooth, comprising identifying a tooth having a filling space in need of filling, positioning a curable mixture within the filling space, and curing the curable mixture within the filling space without the need for an external energy source or additional curing agent.

Some embodiments described herein generally relate to a method of filling a root canal, comprising identifying a tooth having a root canal in need of filling, positioning a curable mixture within the root canal, and curing the curable mixture within the root canal without the need for an external energy source or additional curing agent.

Some embodiments described herein generally relate to a method of filling a root canal, comprising identifying a tooth having a root canal in need of filling, combining a least a first part and a second part to form a curable mixture, at least one of the first and second parts having a bulk viscosity in the range of about 10 cps to about 200 cps, positioning the curable mixture within the root canal, and exposing the curable mixture within the root canal to water for a period of time effective to cure the curable mixture. In some embodiments, the water that facilitates the cure of the curable mixture is from the curable mixture, bodily fluids or both. In some embodiments, the curable mixture is substantially anhydrous, and the water is from bodily fluids. In some embodiments, positioning the filling material within the root canal comprises expressing at least one component at a rate that is effective to flow the curable mixture into a filling hole in the tooth.

These and other embodiments are described in greater detail below.

### BRIEF DESCRIPTION OF THE DRAWINGS

The foregoing and other features, aspects, and advantages of the embodiments of the apparatus, compositions and methods of filling spaces in teeth are described in detail below with reference to the drawings of various embodiments, which are intended to illustrate and not to limit the embodiments of the invention. The drawings comprise the following figures in which:

FIG. 1A is a schematic diagram of a dental treatment system for treating a root canal, according to various embodiments disclosed herein.

FIG. 1B is a schematic diagram of a system that includes components configured to clean unhealthy or undesirable material from a treatment region on an exterior surface of the tooth.

FIG. 1C is a schematic diagram of the system of FIG. 1B, in which the system is configured to fill a treated carious region of the tooth.

### DETAILED DESCRIPTION

To protect the long-term health of the tooth, it can be advantageous to substantially fill the filling space or spaces of a tooth created from removal of caries, root canal treatment, and/or natural wear. When the restoration follows a root canal treatment it can be important to fill not only the major canal spaces, but also any minor cracks and open spaces in the tooth with the filling material. Similarly, when the restoration follows a caries treatment it can be important to fill the resulting dental spaces in order to provide dimensional stability and/or structural integrity to the tooth.

In various embodiments, the filling material is an obturation material. The term “obturation material” refers to a material that is configured to fill root canals, restore carious lesions, and/or modify the surface of the tooth. The obturation material can be a polymerizable restorative composition that includes a curable mixture that is cured or hardened to form the final material, which may be referred to as a cured mixture or “tooth filling.” Indeed, it should be appreciated that terms such as setting, curing, hardening, polymerizing, etc. all refer to processes by which the obturation material components are transformed into the final cured mixture in the tooth. In this context, an obturation material is “suitable for use as a tooth filling” when the corresponding cured tooth filling has properties that meet standards set by an appropriate regulatory body (e.g. ISO 6876). A cured obturation material having such properties is considered to meet the standards regardless of whether the regulatory body has provided official notification to that effect.

Furthermore, a material having “dimensional stability” refers to a material maintaining its original dimension to a suitable degree for its intended purpose, wherein the “suitable degree” and “intended purpose” for which a dimensionally stable cured obturation material maintains its original dimensions can be informed by an appropriate regulatory body, regardless of whether the regulatory body has provided official notification to that effect. Slight expansion upon setting is also possible, and in some cases preferred, to ensure there is optimal marginal contact and penetration into adjacent open spaces. It should be appreciated that the obturation materials disclosed herein can be used in conjunction with filling root canals after root canal treatments, with filling treated carious regions after treatment, and/or adding to the existing tooth and/or adjacent bone structure either with or without the removal of existing tooth and/or adjacent bone structure. For example, the obturation materials disclosed herein can be used in the manner described in U.S. Pat. No. 9,877,801, the entire contents of which are incorporated herein by reference in their entirety and for all purposes.

In some embodiments, various obturation material compositions or components thereof as described herein can be formed into a coherent collimated jet. For example, in an embodiment, an obturation material composition or components thereof as described herein can be formed into a liquid jet that forms a substantially parallel beam (e.g., is “collimated”) over distances ranging from about 0.01 cm to about 10 cm. In some embodiments, the velocity profile transverse to the propagation axis of the jet is substantially constant (e.g., is “coherent”). For example, in some implementations, away from narrow boundary layers near the outer surface of the jet (if any), the jet velocity is substantially constant across the width of the jet. Therefore, in certain advantageous embodiments, the liquid jet (e.g., as delivered by an apparatus as described herein) may comprise a coherent, collimated jet (a “CC jet”). In some implementations, the CC jet may have velocities in a range from about 100 m/s to about 300 m/s, for example, about 190 m/s in some embodiments. In some implementations, the CC jet can have a diameter in a range from about 5 microns to about 1000 microns, in a range from about 10 microns to about 100 microns, in a range from about 100 microns to about 500 microns, or in a range from about 500 microns to about 1000 microns. Further details with respect to CC jets that can be comprised of obturation material compositions or components thereof as described herein can be found in U.S. Patent Publication No. 2007/0248932, which is hereby incorporated by reference herein in its entirety for all that it discloses or teaches.

In an embodiment, the curable mixture of ingredients comprises:

(a) a polymerizable/cross-linkable polysaccharide;

(b) a divalent cation source;

(c) a filler material; and

(d) a carrier liquid.

In various embodiments, the ingredients (a), (b), (c) and (d) are in the form of multiple parts, such as a two-part curable mixture that comprises a part 1 and a part 2. In an embodiment, the two-part curable mixture is curable by combining part 1 with a coherent collimated jet that comprises part 2 to form a combined curable mixture that comprises the ingredients (a), (b), (c) and (d). In various embodiments, one or both of part 1 and part 2 is degassed. Devices suitable for forming such a coherent collimated jet and carrying out such combining of parts 1 and 2 are described in U.S. Pat. No. 9,877,801. In various embodiments, the ingredients (a), (b), (c) and (d) are selected to provide the combined curable mixture with a viscosity that facilitates delivery of the combined curable mixture into a space in a tooth to form a cured mixture. In some embodiments, the ingredients (a), (b), (c) and (d) are selected to provide the cured mixture with dimensional stability within the space in the tooth.

Component (a), a polymerizable polysaccharide, can comprise alginate, chitosan, pectinate, or mixtures thereof. In some embodiments, component (a) comprises an alginate polymer. In some embodiments, the alginate polymer comprises guluronic units and mannuronic units in a ratio of the guluronic units to the mannuronic units in the range of about 1:1 to about 4:1. In some embodiments, a polymerizable polysaccharide is a cross-linkable polysaccharide.

In some embodiments, the alginate polymer comprises an alginate polymer salt. In some embodiments, the alginate polymer salt comprises an alkali metal cation. In some embodiments, the alginate polymer salt comprises Li alginate, Na alginate, K alginate, or a mixture thereof. Reference herein to an alginate polymer should be understood as encompassing both the polymer and its salt, unless the context indicates otherwise. In some embodiments, the alginate polymer salt comprises Na Alginate.

In some embodiments, the alginate polymer comprises a ratio of the guluronic units to the mannuronic units in the range of about 1.5:1 to about 2.3:1. In some embodiments, the alginate polymer has a weight average molecular weight in the range of about 50 kDa to about 500 kDa. In some embodiments, the alginate polymer has a weight average molecular weight in the range of about 120 kDa to about 220 kDa.

In some embodiments, the molar ratio of the guluronic units to the mannuronic units in the alginate polymer is greater than or equal to any one of 1:1, 1.5:1, 2:1, 2.3:1, 2.5:1, 3:1, 3.5:1 or 4:1. In some embodiments, the molar ratio of the guluronic units to the mannuronic units is in a range that is between any two of the aforementioned ratios. For example, in various embodiments the molar ratio of the guluronic units to the mannuronic units is in the range of about 1:1 to about 4:1, about 1:1 to about 3.5:1, about 1:1 to about 3:1, about 1:1 to about 2.5:1, about 1:1 to about 2.3:1, about 1:1 to about 2:1, about 1.5:1 to about 4:1, about 1.5:1 to about 3.5:1, about 1.5:1 to about 3:1, about 1.5:1 to about 2.5:1, or about 1.5:1 to about 2.3:1.

In some embodiments, the alginate polymer has an average molecular weight in the range of any one of about 50 kDa to about 500 kDa, about 75 kDa to about 300 kDa, about 120 kDa to about 220 kDa, about 150 kDa to about 200 kDa, about 50 kDa to about 300 kDa, or about 75 kDa to about 500 kDa. In some embodiments, molecular weight refers to the weight average molecular weight.

The obturation material mixture can contain various amounts of the alginate polymer. For example, in some embodiments, the amount of alginate polymer in the mixture is in any one of the following ranges: 1-20 wt. %, 5-15 wt. %, 7-12 wt. %, 8-10 wt. %, 1-10 wt. %, or 10-20 wt. % (based on total weight of mixture). In some embodiments, the mixture comprises alginate in any one of the amounts within the aforementioned ranges, such as about 7 wt. %, about 8 wt. %, about 9 wt. %, about 10 wt. %, or about 11 wt. % alginate.

In some embodiments, the alginate polymer is in a microparticulate form. In some embodiments, the microparticles have an average particle size of about 5 microns or less, about 3 microns or less, or about 2 microns or less. In some embodiments, the alginate polymer is substantially anhydrous.

Component (b), a divalent cation source, can comprises an element such as Ca, Ba, Sr, or a mixture thereof. In some embodiments, the divalent cation source can be an ionic material that comprises a metal salt. Examples of metal salts include, but are not limited to Ca(OH)₂, Ba(OH)₂, Sr(OH)₂, CaSO₄, BaSO₄, SrSO₄, CaCl₂, BaCl₂, SrCl₂, calcium gluconate, calcium citrate, calcium carbonate, barium gluconate, barium citrate, barium carbonate, strontium gluconate, strontium citrate, strontium carbonate, or a mixture thereof. In some embodiments, the ionic material is SrSO₄.

A divalent cation ionic material can be used to deliver a divalent cation to the alginate polymer. Particularly in the case of alginate polymer salts having monovalent counterions, divalent cations (such as calcium or strontium) tend to displace the monovalent counterions (such as sodium and/or potassium) due to the alginate's greater affinity for divalent cations. These divalent cations allow for crosslinking of the alginate polymer strands resulting in the formation of hydrogels. Furthermore, divalent cation alginate salts are generally insoluble in water, as will be discussed further below.

Various ionic materials that comprise a divalent cation can be included in the curable mixture. In some embodiments, the divalent cation is present in the curable mixture in an amount effective to crosslink the alginate polymer upon exposure of the mixture to an effective amount of water. For example, for an anhydrous curable mixture in which the ionic material is insoluble, exposure to water solubilizes at least a portion of the ionic material to thereby allow the divalent cation to react with and crosslink the alginate polymer. In some embodiments, the divalent cation comprises an element selected from Ca, Ba, Sr, or a mixture thereof. In some embodiments, the divalent cation is Ca. In some embodiments, the divalent cation is Ba. In some embodiments, the divalent cation is Sr.

The curable mixture can contain various amounts of the ionic material, which can be in the form of a metal salt. In some embodiments, the mixture comprises an amount of metal salt in any one of the following ranges, such as 1-10 wt. %, 2-7 wt. %, 3-5 wt. %, 1-5 wt. %, or 3-10 wt. % ionic material. For example, in some embodiments, the mixture comprises about 3 wt. %, about 4 wt. %, or about 5 wt. % ionic material.

Component (c), a filler material, can comprise but is not limited to inorganic metal, salt, oxide, fluoride, silicate glass, quartz, or mixtures thereof.

Fillers can be used to adjust the viscosity and/or rheological properties of the curable mixture. The curable mixture can comprise various filler materials, which in some embodiments are non-reactive with tooth material and/or the other components of the mixture. For example, in some embodiments, the mixture comprises a non-reactive filler. In other embodiments, the mixture comprises a filler material that is an inorganic material such as ZnO, a bioactive glass, fumed silica, a non-reactive glass, a diatomaceous earth, or a mixture thereof. In some embodiments, the filler material is ZnO. In some embodiments, the filler material is fumed silica. Examples of fumed silica include, but is not limited to, Aerosil OX-50, Aerosil OX-130, Aerosil OX-200, Cab-O-Sil TS530, Cab-O-Sil TS720 and Cab-O-Sil M5, and mixtures therof. In some embodiments, the filler material is a mixture of ZnO and fumed silica. In some embodiments, the filler material is a bioactive glass. In some embodiments, the bioactive glass is a calcium containing glass such as Bioglass. In some embodiments, the filler material is a non-reactive glass. Examples of non-reactive glass include, but are not limited to, bariumaluminosilicate, bariumborosilicate, bariumaluminoborosilicate, strontiumaluminosilicate, strontiumborosilicate and strontiumaluminoborosilicate, and mixtures thereof. In some embodiments, the non-reactive glass is bariumborosilicate glass.

Various amounts of filler material can be included in the curable mixtures described herein, depending on the viscosity and/or rheological properties desired. In some embodiments, the mixture comprises an amount of a filler material in any one of the following ranges, such as 0.1-10 wt. %, 0.5-5 wt. %, 1-3 wt. %, 0.1-3 wt. %, 1-10 wt. %, 2-6 wt. %, or 1-8 wt. % filler material. For example, in some embodiments, the mixture comprises a filler material in an amount of about 1 wt. %, about 2 wt. %, about 3 wt. %, about 4 wt. %, about 5 wt. %, about 6 wt. %, or about 7 wt. % filler material.

In some embodiments, the filler material is in a microparticulate form. In some embodiments, the microparticles have an average particle size of about 5 microns or less, about 3 microns or less, or about 2 microns or less. In some embodiments, the filler material is substantially anhydrous.

In some embodiments, the non-reactive filler can be an X-ray radiopaque material containing one or more x-ray radiopaque elements. Examples of x-ray radiopaque elements include, but are not limited to, Yb, Ba, Bi, W, Sr, Zr or a mixture thereof. In some embodiments, the X-ray radiopaque material comprises YbF₃, BaF₂, BaSO₄, SrSO₄, BaWO₄, CaWO₄, SrWO₄ or a mixture thereof. In some embodiments, the X-ray radiopaque material is YbF₃. In an embodiment, the curable mixture comprises an X-ray radiopaque material and a filler that is not an X-ray radiopaque material.

Various amounts of the X-ray radiopaque material can be included in the curable mixture. Typically, the amount is selected to be effective to render the resulting cured mixture X-ray radiopaque as defined by the International Standards Organization (e.g., ISO 6876:2012). In some embodiments, the mixture comprises an amount of an X-ray radiopaque material in any one of the following ranges, such as 10-30 wt. %, 15-25 wt. %, 18-22 wt. %, 10-22 wt. %, 18-30 wt. %, or 25-35 wt. %. For example, in some embodiments, the mixture comprises an amount of an X-ray radiopaque material within one or more of the aforementioned ranges, such as about 18 wt. %, about 19 wt. %, about 20 wt. %, about 21 wt. %, about 22 wt. %, or about 30 wt. % X-ray radiopaque material.

In some embodiments, the X-ray radiopaque material is in a microparticulate form. In some embodiments, the microparticles have an average particle size of about 5 microns or less, about 3 microns or less, or about 2 microns or less. In some embodiments, the X-ray radiopaque material is substantially anhydrous.

In some embodiments, the curable mixture comprises a microparticulate filler. In some embodiments, the microparticulate filler material comprises ZnO, a bioactive glass, a non-reactive glass, fumed silica, or a mixture thereof. In some embodiments, the filler material comprises microparticles having an average particle size of about 3 microns or less, wherein the microparticles comprise ZnO, fumed silica, or both. In some embodiments, the filler material comprises microparticles having an average particle size of about 0.1 microns to about 3 microns, wherein the microparticles comprise ZnO, fumed silica, or both.

Component (d), a carrier liquid, comprises a water soluble or water miscible carrier liquid. Examples of carrier liquids are, but are not limited to, water, acetic acid, acetone, acetonitrile, 1-butanol, 2-butanone, ethyl acetate, methanol, ethanol, propanol, butanol, dimethyl sulfoxide, dimethylformamide, 1,4-dioxane, methyl isocyanide, pyridine, tetrahydrofuran, ethylene glycol, propylene glycol, triethylene glycol, glycerol, and mixtures of any two or more of the foregoing. In some embodiments, the carrier liquid is a polyol or mixture of polyols. Examples of polyols include, but are not limited to ethylene glycol, propylene glycol, poly(ethylene glycol), poly(propylene glycol), glycerol and mixtures thereof. In some embodiments, the water soluble or water miscible carrier liquid comprises glycerol.

In some embodiments, the curable mixture further comprises a water miscible carrier liquid with a viscosity in the range of about 0.1 cps to about 1000 cps. In some embodiments, the water miscible carrier liquid is a mixture having a viscosity in the range of about 1 cps to about 100 cps.

A carrier fluid can dissolve and/or suspend the other ingredients of the curable mixture, so that the mixture can be more conveniently applied to a tooth. In some embodiments, the carrier liquid is water soluble. In some embodiments, the carrier liquid is water miscible. In some embodiments, the carrier liquid is substantially anhydrous. In some embodiments, the carrier liquid comprises water. The mixture can contain a variety of carrier liquids or mixtures of carrier liquids. For example, in some embodiments, the carrier liquid can comprise a polyol. In some embodiments, the polyol can be selected from ethylene glycol (EG), propylene glycol (PG), poly(ethylene glycol), diethylene glycol (DEG), poly(propylene glycol), glycerol (Gly) and mixtures thereof. In an embodiment, the polyol is glycerol, which may also be referred to as glycerin. In another example, in some embodiments, the carrier liquid can be selected from water, ethylene glycol (EG), propylene glycol (PG), poly(ethylene glycol), poly(propylene glycol), diethylene glycol (DEG), ethanol (EtOH), glycerol (Gly) and mixtures thereof. In some embodiments, the carrier liquid comprises glycerol and one or more additional polyols. In some embodiments, the one or more additional polyols is a polyol with a molecular weight of less than 155 g/mol, less than 150 g/mol, less than 130 g/mol, or less than 120 g/mol. In some embodiments, the one or more additional polyol comprises ethylene glycol, diethylene glycol, or a combination thereof.

In some embodiments, a first carrier liquid can be applied to the dry ingredients of the curable mixture to form a paste, and then a second carrier liquid can be applied to the paste to form a curable mixture suitable for filling a tooth space as described elsewhere herein. The use of a first carrier liquid and a second carrier liquid can enable more convenient application of the curable mixture to a tooth, and better control of material characteristics of each part, such as viscosity and setting time. In some embodiments, at least one of the first carrier liquid and second carrier liquid can comprise water. In some embodiments, at least one of the first carrier liquid and second carrier liquid is substantially anhydrous. In one example, in some embodiments, the final carrier liquid can comprise any one or more selected from water, ethylene glycol (EG), propylene glycol (PG), diethylene glycol (DEG), ethanol (EtOH), glycerol (Gly) and mixtures thereof.

The carrier liquid can be selected on the basis of viscosity in order to effectively apply the mixture to the tooth. In some embodiments, the carrier liquid has a viscosity (e.g., a bulk viscosity) at 25° C. of about 0.5 cps, about 1 cps, about 2 cps, about 3 cps, about 5 cps, about 10 cps, about 15 cps, about 20 cps, about 23 cps, about 24 cps, about 25 cps or about 30 cps, or any range of values therebetween. For example, the carrier liquid can have a viscosity at 25° C. in the range of about 2 cps to about 25 cps.

The curable mixture can contain various amounts of the carrier liquid (which itself may be a mixture). The amount of carrier liquid can be the balance of the weight of the mixture after the amounts of the other ingredients have been specified. For example, if the total of the amounts of the other ingredients (e.g., polymerizable polymer(s) such as a polymerizable/cross-linkable polysaccharide, cross-linking or activating agent(s) such as a divalent cation source, filler material(s), and/or X-ray radiopaque material) is 30 wt. % of the mixture, then the amount of the carrier liquid can be the remaining balance, i.e., 70 wt. % of the mixture. In some embodiments, the mixture comprises an amount of carrier liquid in any one of the following ranges, such as 45-60 wt. %, 50-80 wt. %, 60-70 wt. %, 57-69 wt. %, 50-70 wt. %, or 55-80 wt. % carrier liquid.

The curable mixture can further contain an optional surface active agent to facilitate penetration of the uncured or partially cured curable mixture into small spaces within the tooth and/or root canal system. In an embodiment, the curable mixture that contains the surface active agent is substantially anhydrous. Various surface active agents can be included in the mixture. Examples of the surface active agent include, but are not limited to, polysorbates, sorbitan esters, or a mixture thereof. In some embodiments, the polysorbate is selected from polysorbate 20, polysorbate 40, polysorbate 60, polysorbate 80, and mixtures thereof. In some embodiments, the polysorbate is polysorbate 60. In some embodiments, the sorbitan ester is sorbitan sesquioleate. In some embodiments, the surface active agent comprises sorbitan stearate (TEGO).

The curable mixture can contain various amounts of the surface active agent. In some embodiments, the curable mixture comprises a surface active agent in any one of the following ranges, such as about 0 wt. % to about 5 wt. %, about 1 wt. % to about 5 wt. %, about 0 wt. % to about 3 wt. %, about 1 wt. % to about 3 wt. %, or about 0.01 wt. % to about 0.1 wt. %. In some embodiments, the surface active agent is substantially anhydrous.

The curable mixture can further contain a retardant to slow the cure rate of the obturation material, thereby extending the working life and allowing time for the curable mixture to flow into small spaces within the tooth and/or root canal system. In an embodiment, the curable mixture that contains the retardant is substantially anhydrous. Various retardant materials can be included in the mixture. Examples of the retardant include, but are not limited to, a carbonate salt, a phosphate salt, or mixtures thereof. In some embodiments, the phosphate salt is selected from trisodium phosphate, sodium tripolyphosphate, tetrasodium pyrophosphate, sodium pyrophosphate tetrabasic, or mixtures thereof. In some embodiments, the phosphate salt is sodium pyrophosphate tetrabasic, sodium hexametaphosphate, or both. Other examples of retardants include carrier liquids as described herein, including for example ethanol.

The curable mixture can contain various amounts of the retardant. In some embodiments, the mixture comprises an amount of a retardant in any one of the following ranges, such as about 0 wt. % to about 10 wt. %, about 2 wt. % to about 8 wt. %, about 0 wt. % to about 5 wt. %, about 2 wt. % to about 5 wt. %, or about 0.1 wt. % to about 1 wt. %.

In some embodiments, the retardant is in a microparticulate form. In some embodiments, the retardant microparticles have an average particle size of about 5 microns or less, about 3 microns or less, or about 2 microns or less. In some embodiments, the retardant is substantially anhydrous.

In various embodiments obturation materials for use as tooth fillings are formed from a curable mixture of ingredients that, when cured or during a subsequent cure phase, have one or more of several desirable properties. For example, in some embodiments, the obturation material is biocompatible. In some embodiments, the obturation material is x-ray radiopaque. In some embodiments, the curable mixture of ingredients further provides the obturation material with dimensional stability after cure. In some embodiments, the curable mixture of ingredients has minimal or no shrinkage upon setting. In some embodiments, the curable or cured material is readily removed if necessary.

In some embodiments, the curable mixture has a viscosity that facilitates delivery of the obturation material into a cavity or space in need of repair. In some embodiments the cavity is a root canal. In some embodiments, the curable mixture has a viscosity that facilitates delivery of the obturation material to a cavity without requiring the use of an obturation point or other mechanical means to deliver the curable mixture to the base of the cavity. In some embodiments, the curable mixture has a viscosity (e.g., a bulk viscosity) at 25° C. of about 10 cps, about 15 cps, about 20 cps, about 25 cps, about 30 cps, about 50 cps, or about 100 cps, or any range of values therebetween. For example, the curable mixture can have a viscosity at 25° C. in the range of about 10 cps to about 100 cps. In some embodiments, the cavity has a first cross-sectional dimension at the apex of the filling space of about 100 μm, about 125 μm, about 150 μm, about 175 μm, about 200 μm, about 225 μm or about 250 μm, or any range of values therebetween. For example, in an embodiment the cavity has a first cross-sectional dimension at the apex of the cavity in the range of about 150 μm to about 200 μm. In some embodiments, the cavity has a second cross-sectional dimension at a coronal portion of the filling space of about 500 μm, about 600 μm, about 700 μm, about 800 μm, about 900 μm, about 1 mm or about 1.2 mm, or any range of values therebetween. For example, in an embodiment the filling space has a second cross-sectional dimension at a coronal portion of the filling space in the range of about 100 μm to about 4 mm.

In some embodiments, the curable mixture of ingredients comprises one or more parts. In a “one-part” embodiment, the ingredients of the curable mixture are combined together in a single composition. In a “two-part” embodiment, one or more ingredients of the mixture are contained in a first part, one or more ingredients are contained in a second part, and curing commences at a time after the first and second parts are combined. Similarly, in a “three-part” embodiment, one or more ingredients of the mixture are contained in a first part, one or more ingredients are contained in a second part, one or more ingredients of the mixture are contained in a third part, and curing commences at a time after the first, second and third parts are combined. A curable mixture as described herein will thus be understood to be in the form of one, two, three or more parts, unless the context indicates otherwise. In some embodiments, the one or more parts of the mixture can be expressed through an opening of less than, or about, 10 μm, 50 μm, 60 μm, 80 μm, 100 μm, 150 μm, 200 μm, or any range of values therebetween. For example, in an embodiment one or more parts can each individually be expressed through an opening in the range of about 50 μm to about 150 μm, or less than 100 μm.

In some embodiments, the obturation material comprises an alginate polymer, as described herein. In some embodiments, the curable mixture comprises a divalent cation ionic material, as described herein.

In some aspects, the present disclosure describes an advanced curable mixture comprising an alginate polymer. The various ingredients of the mixture are selected to provide a curable mixture. In various embodiments, the obturation material mixture is cured by exposure to water and thus the uncured or curable mixture can be substantially anhydrous in order to prolong shelf and operatory working time. In this context, the term “substantially anhydrous” refers to an uncured obturation material mixture that, in the absence of moisture, does not exhibit curing for a period of at least 12 hours. For example, in various embodiments the substantially anhydrous curable mixture contains less than 1 wt. % water, less than 0.5 wt. % water or less than 0.1 wt. % water. In various other embodiments, water is added to form the obturation material mixture and thus initiate curing of the curable mixture.

Various beneficial characteristics of embodiments of the curable mixture are described herein. In some embodiments, the ingredients of the mixture are selected to provide properties suitable for use as a tooth filling after curing by exposure of the mixture to an effective amount of water. Curing of the curable mixture by exposure to water can enable the curable mixture to be cured after it is applied to the tooth, as moisture inside a patient's own mouth can be used to cure the mixture. Additionally, an external water source can also be used to cure or to assist cure of the curable mixture. For example, in some embodiments, water can be added to form the uncured obturation material mixture before or as the mixture is applied to the tooth.

In some embodiments, the curable mixture has a viscosity that is effective to permit flow into the complex anatomy of a tooth. For example, in some embodiments, the curable mixture has a viscosity effective to permit flow into the complex anatomy of a tooth, wherein the tooth comprises a filling space with a diameter as described elsewhere herein. For example, in some embodiments, the filling space diameter is a cross-sectional dimension at the apex of the space, as described elsewhere herein. In another example, in some embodiments, the filling space diameter is a cross-sectional dimension at a coronal portion of the space, as described elsewhere herein. In some embodiments, the curable mixture is suitable for use as a root canal filling after curing by exposure of the curable mixture to an effective amount of water.

In various embodiments, the curable mixture comprises effective amounts of a carrier liquid that is substantially water free, an alginate polymer, an ionic material that comprises a divalent cation, a filler material, and an X-ray radiopaque material, as described in greater detail herein. In various other embodiments, the curable mixture comprises effective amounts of a first carrier liquid that is substantially water free, a second carrier liquid comprising >1% water, an alginate polymer, an ionic material that comprises a divalent cation, a filler material, and an X-ray radiopaque material, as described in greater detail herein.

Numerous alginate polymers are useful as ingredients for the obturation material mixtures described herein. In various embodiments, alginate polymers comprise guluronic units and mannuronic units. Alginate polymers can have different characteristics based on a multitude of factors, such as the molar ratio of the guluronic units to the mannuronic units, the molecular weight of the polymer and the ions bonded to the polymer if it is in a salt form. In some embodiments, molecular weight refers to the weight average molecular weight.

Some embodiments described herein generally relate to a substantially anhydrous curable mixture, comprising a mixture that comprises at least the following ingredients:

(a) an alginate polymer comprising guluronic units and mannuronic units,

(b) an ionic material that comprises a divalent cation, the divalent cation being present in an amount effective to crosslink the alginate polymer upon exposure of the curable mixture to an effective amount of water,

(c) an X-ray radiopaque material, and

(d) a water soluble or water miscible carrier liquid.

In some embodiments, the ingredients (a), (b), (c), and (d) are substantially anhydrous. In some embodiments, the alginate polymer comprises a ratio of the guluronic units to the mannuronic units in the range of about 1:1 to about 4:1. In some embodiments, the ingredients (a), (b), (c), and (d) are selected to provide the substantially anhydrous curable mixture with properties suitable for use as a tooth filling after curing by the exposure of the curable mixture to an effective amount of water.

In some embodiments, the ingredients (a), (b), (c), and (d) are selected to provide the substantially anhydrous curable mixture with a viscosity effective to permit flow into the complex anatomy of a tooth. In some embodiments, the tooth comprises a filling space with a diameter of about 150 μm to about 200 μm. In some embodiments, the tooth comprises a filling space with a diameter in the range of about 150 μm to about 200 μm at the base. In some embodiments, the ingredients (a), (b), (c), and (d) are selected to provide the substantially anhydrous curable mixture with properties suitable for use as a root canal filling after curing by the exposure of the curable mixture to the effective amount of water.

In some embodiments, the substantially anhydrous curable mixture comprises 8-10 wt. % sodium alginate, 3-5 wt. % CaSO₄, 1-4 wt. % ZnO, 1-3 wt. % fumed silica, 18-30 wt. % YbF₃, and the balance comprising glycerol and, optionally, a surface active agent and/or a retardant. In some embodiments, the substantially anhydrous curable mixture comprises 7-15 wt. % sodium alginate; 3-5 wt. % SrSO₄; 2-8 wt. % ZnO; 1-3 wt. % fumed silica; 18-30 wt. % YbF₃; and the balance comprising glycerol and, optionally, a surface active agent and/or a retardant. In some embodiments, the substantially anhydrous curable mixture comprises 7-15 wt. % sodium alginate; 3-5 wt. % CaSO₄; 2-8 wt. % ZnO; 1-3 wt. % fumed silica; 18-30 wt. % YbF₃; and the balance comprising glycerol and, optionally, a surface active agent and/or a retardant.

In some embodiments, the curable mixture comprises one or more ingredients that can be expressed through an opening or an orifice of less than 100 μm. In various embodiments, the ingredients and the final curable mixture are stable and can function in temperatures minimally between 0° C. and 50° C.

In some embodiments, the curable mixture has a viscosity that facilitates delivery of the curable mixture into a filling space in the tooth at a temperature of 37° C., the filling space having a diameter in the range of about 150 μm to about 200 μm at an apex of the filling space. In some embodiments, the curable mixture comprises one or more materials that can be expressed through an opening of less than 100 μm.

Alginate Mixture Embodiments

In some embodiments, the curable mixture comprises about 8-10 wt. % sodium alginate; 3-5 wt. % CaSO₄; 1-4 wt. % ZnO; 1-3 wt. % fumed silica; 18-30 wt. % YbF₃; and the balance comprising glycerol and, optionally, a surface active agent and/or a retardant. In some embodiments, the mixture comprises about 7-15 wt. % sodium alginate; 3-5 wt. % SrSO₄; 2-8 wt. % ZnO; 1-3 wt. % fumed silica; 18-30 wt. % YbF₃; and the balance comprising glycerol and, optionally, a surface active agent and/or a retardant. In some embodiments, the mixture comprises about 7-15 wt. % sodium alginate; 3-5 wt. % CaSO₄; 2-8 wt. % ZnO; 1-3 wt. % fumed silica; 18-30 wt. % YbF₃; and the balance comprising glycerol and, optionally, a surface active agent and/or a retardant.

### Alginate Curing

In some embodiments, the curable mixture can be hardened by mixing with water or due to moisture inside a tooth, a root canal system, or a treatment region. As divalent alginate salts are generally insoluble in water, the presence of water can facilitate the reaction between the alginate polymer and the divalent cation of the ionic material to form the resulting insoluble divalent alginate salt and thereby facilitating the hardening (cure) of the obturation material. The amount of water effective to facilitate reaction of the divalent cation with the alginate polymer can be determined by routine experimentation informed by the guidance provided herein.

In some embodiments, a mixture can be hardened or cured without the need for an external energy source. In some embodiments, a mixture can be hardened or cured without the need for an additional curing agent.

### Methods of Use

The curable mixtures described herein can be used in various methods of filling a tooth. In some embodiments, the method comprises positioning an obturation material within a cavity. In some embodiments, the method comprises exposing the substantially anhydrous curable mixture within the cavity to water for a period of time effective to cure the mixture.

In some embodiments, the curable mixture can be used in a method of filling a root canal. In some embodiments, the method comprises positioning an obturation material within the root canal. In some embodiments, the method comprises exposing a substantially anhydrous curable mixture within the root canal to water for a period of time effective to cure the mixture. In some embodiments, the water is from the carrier liquid, bodily fluids, or both.

In some embodiments, the method comprises curing the curable mixture within the cavity without the need for an external energy source or additional curing agent. In some embodiments, the curable mixture is positioned within the root canal by mixing with a carrier liquid that has a bulk viscosity of about 5 cps, about 10 cps, about 20 cps, about 30 cps, about 50 cps, about 75 cps, about 100 cps, about 125 cps, about 150 cps, about 170 cps, about 190 cps, about 200 cps or about 250 cps, or any range of values therebetween. For example, in an embodiment, the carrier liquid can have a bulk viscosity in the range of about 10 cps to about 200 cps.

Curable materials and obturation materials described herein can be formed and applied to a tooth by various methods and devices. The obturation material can be formed in any suitable manner. For example, in some embodiments, a clinician can form the obturation material by mixing the obturation material ingredients, e.g., by hand, by a mechanical tool, or by a mixing device. Furthermore, the obturation material can be applied to a tooth in any suitable manner. For example, in some embodiments, a clinician can apply the obturation material by placing it in the tooth, e.g., by hand, by syringe, by a mechanical tool, or by an application device, such as a device as described in U.S. Pat. No. 9,877,801.

### Application Device

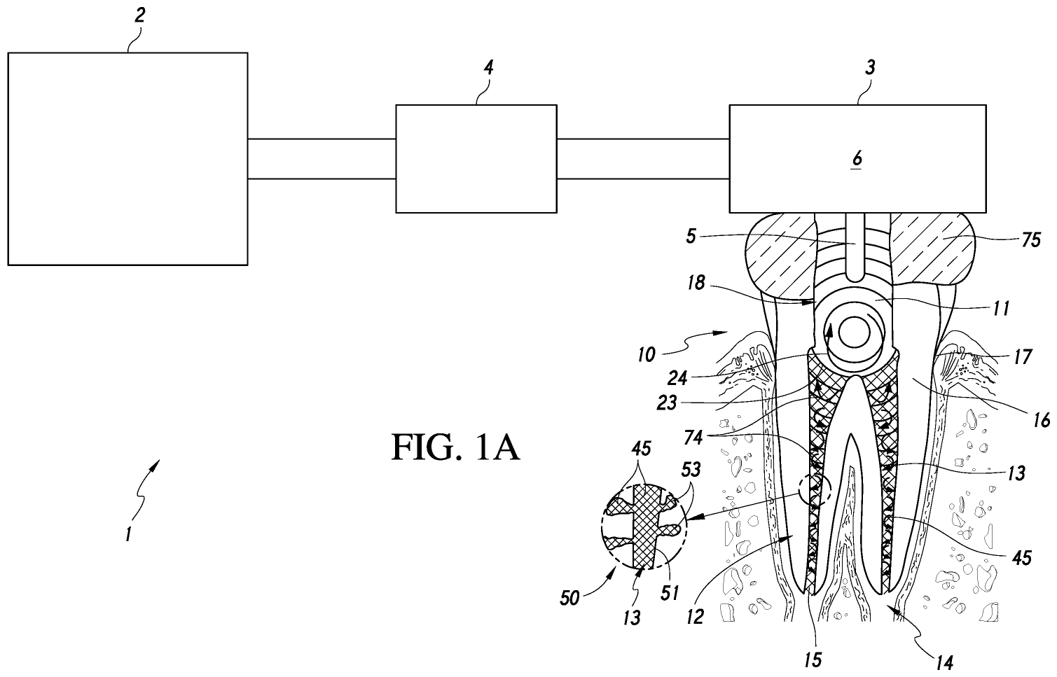

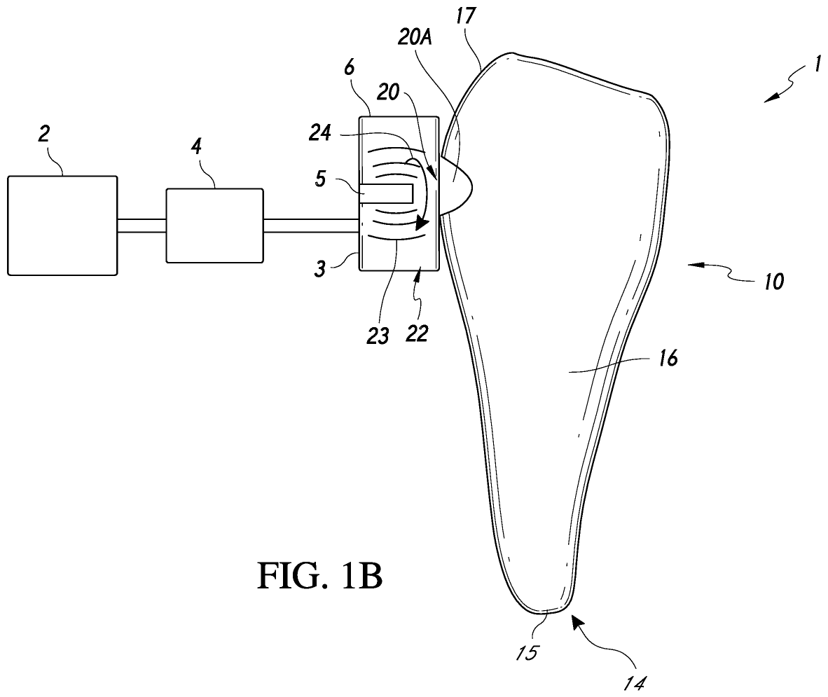

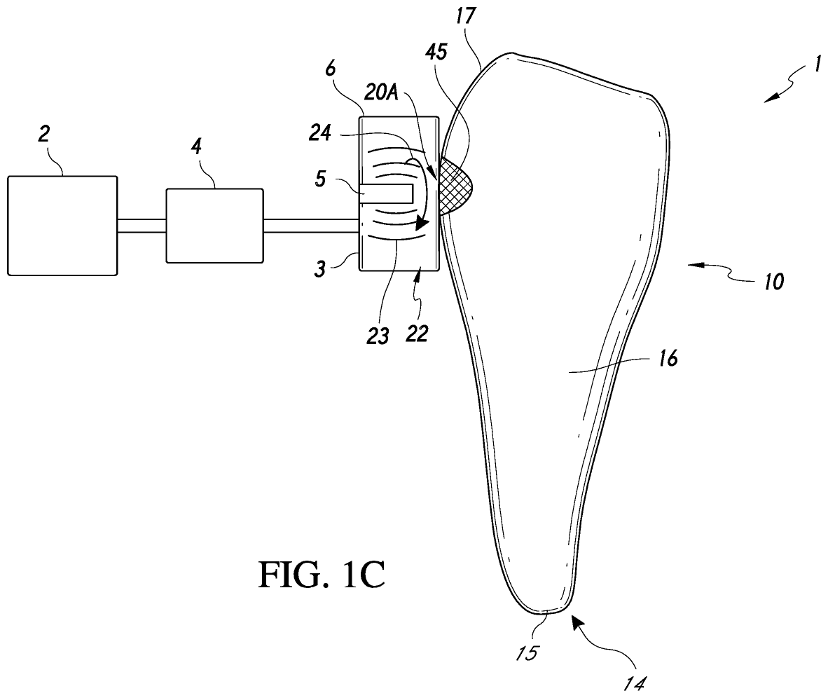

The curable materials and obturation materials described herein can be formed and applied to a tooth by various methods and devices. The filling or obturation material can be formed in any suitable manner. For example, in some embodiments, a clinician can form the obturation material by mixing the obturation material ingredients, e.g., by hand, by a mechanical tool, or by a mixing device. Furthermore, the obturation material can be applied to a tooth in any suitable manner. For example, in some embodiments, a clinician can apply the obturation material by placing it in the tooth, e.g., by hand, by syringe, by a mechanical tool, or by an application device. As described below and in FIGS. 1A-1C, embodiments of a mixing device and/or an application device that can be used to form and/or apply an obturation material are disclosed.

FIG. 1A is a schematic diagram of a system 1, in accordance with embodiments of an application or delivery device as disclosed herein. The system 1 can be configured to perform various types of treatment procedures, including, e.g., cleaning treatments, obturation or other filling treatments, restoration treatments, etc. In the embodiment shown in FIG. 1A, the system 1 is illustrated as being coupled to (e.g., positioned against in some arrangements) a tooth 10 that is a molar tooth of a mammal, such as a human. However, the tooth 10 can be any other suitable type of tooth, such as a pre-molar, bicuspid, incisor, canine, etc. Furthermore, the system 1 shown in FIG. 1A can include components configured to remove unhealthy or undesirable materials from a tooth or surrounding gum tissue, for example, a root canal 13 of the tooth 10. Thus, in the embodiment of FIG. 1A, the system 10 can also be configured to clean the tooth 10, in addition to being configured to fill or obturate the tooth. Moreover, although the treatment shown in FIG. 1A is a root canal treatment, in other embodiments, the application device and obturation material(s) disclosed herein can be used to fill other types of treatment regions, such as a treated carious region of the tooth.

The tooth 10 includes hard structural and protective layers, including a hard layer of dentin 16 and a very hard outer layer of enamel 17. A pulp cavity 11 is defined within the dentin 16. The pulp cavity 11 comprises one or more root canals 13 extending toward an apex 14 of each root 12. The pulp cavity 11 and root canal 13 contain dental pulp, which is a soft, vascular tissue comprising nerves, blood vessels, connective tissue, odontoblasts, and other tissue and cellular components. Blood vessels and nerves enter/exit the root canal 13 through a tiny opening, the apical foramen or apical opening 15, near a tip of the apex 14 of the root 12. It should be appreciated that, although the tooth 10 illustrated herein is a molar, the embodiments disclosed herein can advantageously be used to treat any suitable type of tooth, including pre-molars, canines, incisors, etc.

The system 1 can include a console 2, a pressure wave generator 5, and a tooth coupler 3 (such as a handpiece) adapted to couple to the tooth 10. The tooth coupler 3 can couple to the tooth 10 in any suitable way. In some arrangements, the tooth coupler 3 can be positioned against and/or attach to the tooth 10 by way of a tooth seal 75. For example, the clinician can hold the tooth coupler 3 against the tooth 10 during treatment. In some embodiments, the tooth coupler 3 can define a chamber 6 configured to retain fluid therein, such as a filler or obturation material described herein. In some embodiments, the pulp cavity 11 can define a tooth chamber configured to retain fluid therein. In some embodiments, the tooth coupler 3 may not define a chamber, and the tooth chamber defined at least in part by the pulp cavity 11 can retain fluid.

The tooth coupler 3 disclosed herein can be any suitable structure or housing configured to couple to the tooth 10 for a treatment procedure. As used herein, “couple” is meant to include arrangements in which there is a connection with the tooth 10, as well as arrangements in which the coupler 3 is placed against or in the tooth and is held by the clinician in that position. The pressure wave generator 5 can be coupled to and/or disposed in or on the tooth coupler 3 in various embodiments.

A system interface member 4 can electrically, mechanically, and/or fluidly connect the console 2 with the tooth coupler 3 and pressure wave generator 5. For example, in some embodiments, the system interface member 4 can removably couple the tooth coupler 3 to the console 2. In such embodiments, the clinician can use the tooth coupler 3 one time (or a few times), and can dispose the tooth coupler 3 after each procedure (or after a set number of procedures). The console 2 and interface member 4 can be reused multiple times to removably couple (e.g., to connect and/or disconnect) to multiple tooth couplers 3 using suitable engagement features, as discussed herein. The interface member 4 can include various electrical and/or fluidic pathways to provide electrical, electronic, and/or fluidic communication between the console 2 and the tooth coupler 3. The console 2 can include a control system and various fluid and/or electrical systems configured to operate the pressure wave generator 5 during a treatment procedure. The console 2 can also include a management module configured to manage data regarding the treatment procedure. The console 2 can include a communications module configured to communicate with external entities about the treatment procedures.

Additionally, the console 2 can include a control system comprising a processor and non-transitory memory. Computer-implemented instructions can be stored on the memory and can be executed by the processor to assist in controlling cleaning and/or filling procedures. Additional details of the console 2 can be found in U.S. Pat. No. 9,504,536, and in U.S. Pat. No. 9,675,426, each of which is incorporated by reference herein in its entirety and for all purposes.

In FIG. 1A, the system 1 is used to fill or obturate the root canal 13 with an obturation material 45, which can be the same as or generally similar to the filler materials described herein. For example, the clinician can clean the root canal 13 in any suitable way, such as by using drills or files, or by using a pressure wave generator (which can be the same as or different from the pressure wave generator 5 shown in FIG. 1A). When the root canal 13 is cleaned, the clinician can supply an obturation material 45 in its flowable state to the pulp cavity 11, canals 13, or other internal chambers of the tooth 10.

As explained herein, the clinician can supply the obturation material 45 to the treatment region (e.g., the root canal) in any suitable manner. For example, in some embodiments, the pressure wave generator 5 (which can be coupled to or formed with a handpiece) can have one or more openings configured to deliver the flowable obturation material 45 to the tooth 10. In other embodiments, the clinician can supply the obturation material 45 to the tooth by manually placing it in the tooth 10, e.g., by hand, by syringe, or by a mechanical tool. In still other embodiments, a dental handpiece can include one or more supply lines that are configured to route the flowable obturation material 45 to the tooth 10. The obturation material 45 can be any suitable obturation material disclosed herein. In particular, the obturation material 45 can have a flowable state in which the obturation material 45 flows through the treatment region to fill the root canals 13 and/or pulp cavity 11. The obturation material 45 can have a hardened state in which the obturation material 45 solidifies after filling the treatment region.

Advantageously, the pressure wave generator 5 can be activated to enhance the obturation or filling procedure. For example, the pressure wave generator 5 can be activated to assist in flowing the obturation material 45 throughout the treatment region to be filled. The pressure wave generator 45 can thereby assist in substantially filling the tooth 10. As shown in inset 50 of FIG. 1A, for example, when activated, the pressure wave generator 5 can cause the obturation material 45 to flow into major canal spaces 51 of the tooth 10, as well as into small spaces 53 of the tooth 10. Thus, the system 1 shown in FIG. 1A can assist in filling even small cracks, tubules, and other tiny spaces (e.g., the small spaces 53) of the tooth 10. By filling the small spaces 53 of the tooth, the system 1 can ensure a more robust obturation procedure which results in long-term health benefits for the patient. As explained herein, the pressure waves 23 and/or fluid motion 24 (which can include vortices 74) generated by the pressure wave generator 5 can interact with the obturation material 45 to assist in filling the small spaces 53 and the major spaces 51 of the tooth 10. Furthermore, in some embodiments, the pressure wave generator 5 can be activated to assist in curing or hardening the obturation material 45. For example, as explained herein, some types of obturation materials can cure or harden (or the curing or hardening can be enhanced) when agitated by pressure waves 23 generated by the pressure wave generator 5. In addition, in various embodiments, the obturation or filling material can be degassed, which can help deliver the obturation material to small spaces of the tooth. Accordingly, the pressure wave generator 5 can enhance the obturation procedure in a variety of ways.

In some embodiments, the obturation material 45 is supplied to the tooth 10, and the pressure wave generator 5 is subsequently activated to enhance the obturation procedure (e.g., to improve the filling process and/or to enhance or activate the curing process). For example, in such embodiments, the clinician can supply the obturation material 45 to the tooth 10 using a syringe or other device, and the pressure wave generator 5 can subsequently (or concurrently) be activated to fill the treatment region. In other embodiments, the pressure wave generator 5 can supply the obturation material 45 and generate pressure waves through the obturation material (or other fluids at the treatment region). In some embodiments, supplying the obturation material and generating pressure waves can occur substantially simultaneously, or can overlap by some amount over time. For example, the pressure wave generator 5 can be activated to supply the obturation material 45 to the treatment region. For example, in embodiments in which the pressure wave generator 5 comprises a liquid jet, a jet of obturation material 45 can interact with fluids in the tooth 10 (e.g., other portions of the obturation material or other treatment fluid) to generate pressure waves that propagates through the fluids. The resulting pressure waves can enhance the obturation procedure. In various embodiments, the pressure waves can have a broadband of multiple frequencies, which can further enhance the filling of the treatment region. Additional details regarding the generation of broadband pressure waves is shown and described at least in FIGS. 2A-2C, and the associated disclosure, of U.S. Pat. No. 9,877,801, the entire contents of which are incorporated by reference in their entirety and for all purposes. In other embodiments, different types of fluids (e.g., water or other treatment fluids) can form the jet, and the jet can pass through obturation materials in the treatment region. Interaction of the fluid jet and the obturation material can enhance the obturation procedure.

As disclosed herein, the pressure wave generator 5 can comprise any suitable type of pressure wave generator, e.g., a liquid jet device, a laser, a mechanical stirrer, an ultrasonic transducer, etc. The pressure wave generator 5 can be sized such that the pressure wave generator 5 is disposed outside the region of the tooth 10 that is to be obturated. For example, the pressure wave generator 5 can be disposed in the chamber 6 such that it is disposed outside the tooth 10. In other arrangements, the pressure wave generator 5 can extend partially into the tooth 10. In some arrangements, the pressure wave generator 5 can extend to a depth that does not interfere with the filling. The system 1 can include a cleaning mode for cleaning the treatment region and a filling mode to fill or obturate the treatment region. The console 2 can include a control system comprising a processor and memory. The control system can be programmed or configured to switch the system 1 from the cleaning mode to the filling mode and vice versa. The control system of the console 2 can also control the operation of cleaning and/or filling procedures. Additional details of the delivery device shown in FIG. 1A can be found throughout U.S. Pat. No. 9,877,801, the entire contents of which are incorporated herein by reference and particularly for the purpose of describing such details.

FIG. 1B is a schematic diagram of a system 1 that includes components configured to clean unhealthy or undesirable material from a treatment region 20 on an exterior surface of the tooth 10. For example, as in FIG. 1A, the system 1 can include a tooth coupler 3 and a pressure wave generator 5. The tooth coupler 3 can communicate with a console 2 by way a system interface member 4. Unlike the system 1 of FIG. 1A, however, the tooth coupler 3 is coupled to (e.g., positioned against by a clinician) a treatment region 20 on an exterior surface of the tooth 10. In some embodiments, the tooth coupler 3 can be stably positioned against the treatment region and can be sealed to the tooth 10, e.g., by way of an adhesive or other seal. The system 1 of FIG. 1B can be activated to clean an exterior surface of the tooth 10, e.g., a carious region of the tooth 10 and/or remove undesirable dental deposits, such as plaque, calculus biofilms, bacteria, etc, from the tooth 10 and/or surround gum tissue. In other embodiments (see FIG. 1C), the system 1 can be activated to fill a treated region on the exterior surface of the tooth 10 with a filling or restoration material. As with the embodiment of FIG. 1A, pressure waves 23 and/or fluid motion 24 can be generated in the tooth coupler 3 and chamber 6, which can act to clean the treatment region 20 of the tooth 10, forming a cleaned treatment region 20A in which the carious (or other unhealthy material) is removed. Additional details of systems and methods for treating various regions of teeth can be found in International Application Publication WO 2013/142385 (PCT/US2013/032635), having an international filing date of Mar. 15, 2013, entitled “APPARATUS AND METHODS FOR CLEANING TEETH,” the entire contents of which are incorporated by reference herein in their entirety and for all purposes. Additional details of systems and methods for removing undesirable dental deposits (such as plaque, calculus, etc.) from teeth and/or gums can be found in International Application Publication WO 2013/155492 (Application No. PCT/US2013/036493), having an international filing date of Apr. 12, 2013, entitled “APPARATUS AND METHODS FOR CLEANING TEETH AND GINGIVAL POCKETS,” and in U.S. Patent Publication No. US 2014/0099597, filed Apr. 11, 2013, entitled “APPARATUS AND METHODS FOR CLEANING TEETH AND GINGIVAL POCKETS,” each of which is incorporated by reference herein in its entirety and for all purposes.

FIG. 1C is a schematic diagram of the system 1 of FIG. 1B, in which the system 1 is configured to fill the treated carious region 20A of the tooth 10, and can be used in combination with any of the filling materials disclosed herein. As with the embodiment of FIG. 1B, the system can include a pressure wave generator 5, a tooth coupler 3, an interface member 4, and a console 2. When the carious or other unhealthy material is removed from the tooth 10, the clinician can fill the cleaned treatment region 20A with a suitable filler or obturation material 45. As with the embodiment of FIG. 1A, the obturation material 45 can be supplied to the cleaned treatment region 20A. The pressure wave generator 5 can act to substantially fill the treatment region 20A and/or to enhance or activate the hardening of the filler obturation material 45. In some embodiments, the filler or obturation material 45 is supplied to the tooth 10, and the pressure wave generator 5 is subsequently activated to enhance the filling procedure (e.g., to improve the filling process and/or to enhance or activate the curing process). For example, in such embodiments, the clinician can supply the filler or obturation material 45 to the treatment region 20A using a syringe, and the pressure wave generator 5 can subsequently be activated to fill the treatment region. In other embodiments, the pressure wave generator 5 is activated to supply the filler or obturation material 45 to the treatment region 20A and to generate pressure waves through the material. For example, in embodiments in which the pressure wave generator 5 comprises a liquid jet, a jet of obturation or filler material 45 (or other type of fluid) can interact with fluids at the treatment region 20A (e.g., other portions of the filler or obturation material or other treatment fluid) to generate pressure waves that propagates through the fluids. The resulting pressure waves can enhance the obturation procedure.

Although the examples shown in FIGS. 1A-1C describe the delivery device as including a pressure wave generator, it should be appreciated that the obturation material(s) described herein can be used in conjunction with any other suitable type of delivery device. For example, the obturation material(s) described herein can be used with a syringe, a mechanical instrument, or any other suitable device.

### EXAMPLES

### Comparative Example 1: Partially Hardening Alginate Formulation

The ingredients in an obturation material formulation comprising alginate are listed in Table 1 below. The dry ingredients of the formulation were mixed into a homogenous powder, and water was subsequently mixed into the powder as a curing agent. The formulation of Table 1 did not fully harden over the course of the experiment after the addition of water. These results demonstrate that this formulation does not have properties suitable for use as a tooth filling after curing by the exposure of the mixture to the effective amount of water.

TABLE 1

| Ingredient | Amount | Results |
|---|---|---|
| Sodium Alginate (Sigma- | 1.5 g | Paste did not fully harden |
| Aldrich ®—Brown Algae | | after water was added. |
| medium viscosity) | | |
| Calcium Sulfate | 1.6 g | |
| Sodium Hexametaphosphate | 0.1 g | |
| ZnO | 0.4 g | |
| Barium Borosilicate glass | 6.3 g | |
| Water (mixed after powder | 20 mL | |
| was blended) | | |

### Example 2: Fully Hardening Alginate Formulation

The ingredients in an obturation material formulation comprising alginate are listed in Table 2 below. The formulation of Table 2 is similar to that of Table 1 above, however the alginate of this formulation was sourced from DuPont® and is commercially available as Protanal® LF 200 FTS. The dry ingredients of the formulation were mixed into a homogenous powder, and water was subsequently mixed into the powder as a curing agent. The formulation of Table 2 fully hardened within about 3 min after the addition of water. These results demonstrate that this formulation has properties suitable for use as a tooth filling after curing by the exposure of the mixture to the effective amount of water.

TABLE 2

| Ingredient | Amount | Results |
|---|---|---|
| Sodium Alginate (DuPont ®— | 1.5 g | Material fully hardened |
| Protanal ® LF 200 FTS) | | in about 3 min after water |
| Calcium Sulfate | 1.6 g | was added. |
| Sodium Hexametaphosphate | 0.1 g | |
| ZnO | 0.4 g | |
| Barium Borosilicate glass | 6.3 g | |
| Water (mixed after powder | 20 mL | |
| was formed) | | |

### Example 3: Substantially Anhydrous Alginate Formulation

The ingredients of a substantially anhydrous curable mixture formulation comprising alginate and a water miscible carrier liquid are listed in Table 3 below.

TABLE 3

| Ingredient | Description | Amount (wt. %) |
|---|---|---|
| Sodium Alginate | Alginate Polymer | 9% |
| Glycerol | Water Miscible | 51% |
| | Carrier Liquid | |
| CaSO₄ | Ionic Material | 3% |
| Sodium Pyrophosphate | Retardant | <1% |
| Polysorbate 60 | Surface Active | 0.01% |
| | Agent | |
| ZnO | Filler Material | 4% |
| Fumed Silica | Filler Material | 2.7% |
| YbF₃ | X-ray Radiopaque | 30% |
| | Material | |

### Example 4: Stability of Sr²⁺ and Ca²⁺ Cation-Based Alginate Gels in Citric Acid Solutions

Formulations were made with high molecular weight sodium alginates (Protanal LF 200) and low molecular weight sodium alginates (Protanal LF 10/60). Strontium sulfate, calcium sulfate and barium sulfate (sources of divalent cation) were used as cationic gelation (curing) agents, as shown in Table 4.

TABLE 4

| | Formulation, wt % | | | |
|---|---|---|---|---|
| Component | 1 | 2 | 3 | 4 |
| Calcium Sulfate | 3 | 0 | 0 | 0 |
| Barium Sulfate | 0 | 0 | 15 | 0 |
| Strontium Sulfate | 0 | 3 | 0 | 3 |
| Sodium pyrophosphate | 1 | 0 | 0 | 0 |
| Zinc Oxide | 2 | 2 | 3 | 2 |
| Fumed Silica | 3 | 2 | 2 | 1 |
| Barium Borosilicate | 5 | 5 | 2 | 5 |
| Glass | | | | |
| Zirconium Oxide | 0 | 16 | 20 | 16 |
| Ytterbium Fluoride | 28 | 15 | 0 | 16 |
| Sodium Alginate, | 7 | 7 | 7 | 0 |
| Protanal LF 200 FTS | | | | |
| Sodium Alginate, | 0 | 0 | 0 | 7 |
| Protanal LF 10/60 | | | | |
| Glycerol | 51 | 51 | 51 | 50 |
| Polysorbate 60 | 0.09 | 0.1 | 0.1 | 0.1 |
| Results | Set | Set | Did Not | Set |
| | Overnight | Overnight | Set | Overnight |
| | | | Overnight | |

Formed pastes were milled and stored in a sealed container in a dry place. To prepare solid discs, the pastes were placed in gypsum molds incorporating a cavity with a diameter of 10 mm and a height of 1 mm. The gypsum molds were preconditioned at 37° C. and >95% RH for a minimum of 24 h. After filling, the cavities were covered with a glass slide and kept overnight in a humid chamber of >95% RH at 37° C. Advantageously, solid discs were formed for Formulations 1, 2 and 4 of Table 4. The Formulation 3 paste, which comprised barium sulfate, did not set overnight although the quantity of cation used was relatively higher (15% weight) than the amount employed in Formulations 1, 2, and 4. It is believed that Formulation 3 was slow to set due to the low solubility of barium sulfate in water.

Prepared discs of Formulations 1, 2 and 4 of Table 1 were soaked in 30 mL of following solutions and stored at 37° C.: 1) a 7.2 pH phosphate-buffered saline (PBS) buffer; or 2) a sodium citrate solution. The sodium citrate solution was prepared as follows: 0.36 g of sodium citrate monobasic, and 1.29 g of trisodium citrate salts dissolved in 130 mL of distilled water. The pH of the sodium citrate solution was measured as pH=5.79. Stability and disintegration of discs were monitored visually.

After a few hours, discs in citrate solution began swelling. The calcium based Formulation 1 began releasing solid residue, and after 24 hours the disc was fully disintegrated. Unexpectedly and advantageously, discs with strontium sulfate, Formulations 2 and 4, remained intact after 3 days with only slight swelling in citric acid solution, indicating dimensional stability after cure.

### Example 5: Carrier Fluid Deliverable Alginate Formulations

A concentrated paste was prepared for delivery to a root canal using a second carrier fluid, which in part would allow for sufficient radio-opacity in the root canal (>3 mm Al). Experimental pastes used in this example were prepared according to Table 5, and contained ca. 70% w/w of solid materials and ca. 30% w/w glycerol as a first carrier solvent.

TABLE 5

| Component | Wt % |
|---|---|
| Calcium Sulfate | 2.58 |
| Zinc Oxide | 7.43 |
| Fumed Silica | 1.09 |
| Barium | 4.96 |
| Borosilicate | |
| Glass | |
| Zirconium | 24.78 |
| Oxide | |
| Sodium | 9.71 |
| Alginate | |
| Ytterbium | 19.63 |
| Fluoride | |
| Glycerol | 29.74 |
| Polysorbate 60 | 0.09 |
| TOTAL | 100.00 |

In combination with water, the following liquids and/or their combinations were examined as second carrier solvents: Propylene glycol (PG); Ethylene glycol (EG); diethylene glycol (DEG); ethanol (EtOH); and glycerol (Gly). Second carrier solvents and pastes were hand-mixed or mixed and delivered by contacting a coherent collimated jet of the carrier liquid with the paste, and evaluated with regard to set time, filling efficacy, radiopacity in plastic and extracted human teeth. Hand mixing proportions were in 43% wt. liquid and 57% wt. paste ratio. Efficacy of mixing by contacting with a coherent collimated jet of the carrier liquid was monitored by x-ray, to minimize the formation of voids and bubbles. Coherent collimated mixing and delivery are carried out using a device as described in U.S. Pat. No. 9,877,801.

Results of various selections of carrier liquid for delivery of the paste of Table 5 to a canal with a paste:second carrier liquid ratio of 57:43% wt. are presented in Table 6. It was unexpectedly and advantageously discovered that suitable methods of action for coherent collimated delivery were achieved with low viscosity carrier liquids, with carrier liquid viscosities preferably less than 20 centipoise (cps). As glycerol has a viscosity that is greater than 700 cps, it was determined that glycerol would be difficult to use successfully as a sole carrier liquid in coherent collimated mixing and delivery applications.

TABLE 6

| | | | | |
|---|---|---|---|---|
| | | Carrier | | |
| | | solvent | | |
| | | viscosity | | |
| | | at room | | |
| | | temp | Hand mix | Machine |
| No. | Carrier solvent | [cps]¹⁾ | outcome²⁾ | delivery³⁾ |

Paste of Formula Shown in Table 5

| | | | | |
|---|---|---|---|---|
| 1 | Water | 1 | Thickens rapidly | n/a |
| 2 | 60 g PG + 39 g EtOH + | n/a | Becomes milky, | n/a |
| | 1 g H₂O | | no gel formation | |
| 3 | 60 g PG + 37 g EtOH + | n/a | Becomes milky, | n/a |
| | 3 g H₂O | | no gel formation | |
| 4 | 30 g PG + 16 g EtOH + | n/a | Becomes milky, | n/a |
| | 4 g H₂O | | no gel formation | |
| 5 | 70 g PG + 10 g EtOH + | n/a | Becomes milky, | n/a |
| | 20 g H₂O | | no gel formation | |
| 6 | 60 g PG + 40 g water | n/a | Becomes milky, | n/a |
| | | | no gel formation | |
| 7 | 40 g PG + 60 g water | n/a | Thickens rapidly | n/a |
| 8 | 25 g PG + 75 g water | n/a | Thickens rapidly | n/a |
| 9 | 30 g PG + 70 g water | n/a | Thickens rapidly | n/a |
| 10 | 50 g DEG + 50 g water | n/a | Slow setting; | n/a |
| | | | forms thin gel | |
| 11 | 40 g DEG + 60 g water | n/a | Thickens rapidly, | n/a |
| | | | forms good gel | |
| 12 | 60 g Gly + 30 g water + | 20 | Starts to set | Material sets |
| | 10 g DEG | | within 2 min | fast; sets in |
| | | | | pulp chamber |
| | | | | before |
| | | | | descending |
| | | | | into canals |
| 13 | 60 g Gly + 15 g DEG + | 25 | Sets within 40 | Carrier liquid |
| | 25 g Water | | min of initial | highly |
| | | | contact | viscous; |
| | | | | difficult to |
| | | | | remove air |
| | | | | bubbles from |
| | | | | canals |
| 14 | 60 g Gly + 10 g EtOH + | 17 | Starts to set | Material sets |
| | 25 g Water | | within 2 min. | fast; sets in |
| | | | Forms gel | pulp chamber |
| | | | | before |
| | | | | descending |
| | | | | into canals |
| 15 | 60 g Gly + 20 g EtOH + | 23 | Thickens rapidly, | Material sets |
| | 20 g Water | | forms brittle gel | fast. |
| 16 | 60 g Gly + 25 g EtOH + | 20 | Slow setting. | Material |
| | 15 g Water | | Forms crumbly | delivered in |
| | | | material. | canals. |
| | | | | Does not |
| | | | | harden or gel. |
| 17 | 75 g EG + 25 g EtOH | 15 | Slow setting; | Material |
| | | | forms thin gel. | delivered in |
| | | | | plastic block |
| | | | | canals, some |
| | | | | air bubbles. |
| | | | | Does not |
| | | | | harden as gel. |
| 18 | 60 g EG + 40 g water | 17 | Sets within 40 | Fill in the |
| | | | min of initial | canal is |
| | | | contact | diluted. |

1% Sodium Pyrophosphate Added as a Retarder to the Paste Shown
in Table 5

| | | | | |
|---|---|---|---|---|
| 19 | 62.5 g gly + 12.5 g | 21 | Starts setting | Highly |
| | EtOH + 25 g water | | within 2 min. | viscous; |
| | | | Forms gel | difficult to |
| | | | | de-bubble |
| | | | | canal |
| 20 | 62% gly + 20% H₂0 + | 27 | Starts setting | Highly |
| | 18% ETOH | | within 2 min. | viscous; |
| | | | | difficult to |
| | | | | flow thru |
| | | | | console |

Calcium Sulfate Replaced with Strontium Sulfate in the Paste Shown
in Table 5

| | | | | |
|---|---|---|---|---|
| 21 | 65% gly + 25% H₂0 + | 24 | Sets within 2 | Highly |
| | 10% ETOH | | min. | viscous; |
| | | | | difficult to |
| | | | | de-bubble |
| | | | | tooth. |
| 22 | 62.5% gly + 25% H₂0 + | 21 | Sets within 2 | Highly |
| | 12.5% ETOH | | min. | viscous; |
| | | | | difficult to |
| | | | | de-bubble |
| | | | | tooth. |
| 23 | 62% gly + 20% H₂0 + | 27 | Sets within 2 | Highly |
| | 18% ETOH | | min. | viscous; |
| | | | | difficult to |
| | | | | de-bubble |
| | | | | tooth. |

¹⁾Viscosity was measured using Brookfield DV2T extra Viscometer.
²⁾Hand mixing performed with a spatula for 20 sec.
³⁾Coherent Collimated Jet Mixing and Jet Delivery performed with delivery liquid jet pressure of: 6000-7000 Psi; EFD pressure for dispense of paste at 60-80 Psi. Paste dispensing pump: Ultimus I dispenser, from Nordson-I EFD Fluid Dispensing Systems.

It was unexpectedly discovered that a carrier liquid comprising ethylene glycol, diethylene glycol and/or glycerol contribute to the formation of solid alginate gels having dimensional stability after cure. Polar solvents such as ethanol, propylene glycol, 2-propanol and acetone were found to have a greater tendency to collapse the alginate salt and reduce the potential for setting. The experiments summarized in Table 6 demonstrate that at least one embodiment (no. 16) of a successful coherent collimated jet mixing and delivery of concentrated glycerol pastes into root canals can be performed with a carrier liquid composition comprising DEG and water.

1. A polymerizable restorative composition, comprising a two-part curable mixture that comprises at least the following ingredients in the form of a part 1 and a part 2:
part 1: (a) a polymerizable polysaccharide, (b) a divalent cation source, and (c) a filler material; and
part 2: (d) a carrier liquid;
wherein the two-part curable mixture is curable by combining part 1 with a coherent collimated jet that comprises a part 2 to form a combined curable mixture that comprises the ingredients (a), (b), (c) and (d);
wherein the ingredients (a), (b), (c) and (d) are selected to provide the combined curable mixture with a viscosity that facilitates delivery of the combined curable mixture into a space in a tooth to form a cured mixture; and
wherein the ingredients (a), (b), (c) and (d) are also selected to provide the cured mixture with dimensional stability within the space in the tooth.

2. (canceled)

3. The polymerizable restorative composition of claim 1, wherein the polymerizable polysaccharide comprises an alginate polymer.

4. The polymerizable restorative composition of claim 3, wherein the alginate polymer comprises guluronic units and mannuronic units in a ratio of the guluronic units to the mannuronic units in the range of about 1:1 to about 4:1.

5. The polymerizable restorative composition of claim 1, wherein the carrier liquid comprises water.

6. The polymerizable restorative composition of claim 1, wherein part 1 further comprises an X-ray radiopaque material that comprises one or more elements selected from the group consisting of Yb, Ba, Bi, W, Sr and Zr.

7. (canceled)

8. The polymerizable restorative composition of claim 1, wherein one or both of part 1 and part 2 is degas sed.

9. (canceled)

10. (canceled)

11. A curable mixture, comprising a curable mixture that comprises at least the following ingredients (a), (b), (c), (d) and (e):
(a) an alginate polymer comprising guluronic units and mannuronic units;
(b) an ionic material that comprises a divalent cation, the divalent cation being present in an amount effective to crosslink the alginate polymer;
(c) a filler material;
(d) a water miscible carrier liquid; and
(e) an X-ray radiopaque material;
wherein the alginate polymer comprises a ratio of the guluronic units to the mannuronic units in the range of about 1:1 to about 4:1; and
wherein the ingredients (a), (b), (c), (d) and (e) are selected to provide the curable mixture with properties suitable for use as a tooth filling after curing by the exposure of the curable mixture to an effective amount of water.

12. The curable mixture of claim 11, wherein the filler material is a microparticulate filler material

13. The curable mixture of claim 11, wherein the water miscible carrier liquid comprises water.

14. The curable mixture of claim 11, wherein the ingredients (a), (b), (c), (d) and (e) are substantially anhydrous.

15. The curable mixture of claim 1, wherein the water miscible carrier liquid comprises a polyol selected from the group consisting of ethylene glycol, propylene glycol, diethylene glycol, poly(ethylene glycol), poly(propylene glycol), glycerol and mixtures thereof.

16. The curable mixture of claim 15, wherein the water miscible carrier liquid comprises glycerol.

17. The curable mixture of claim 11, wherein the divalent cation comprises an element selected from the group consisting of Ca, Ba, Sr, and a mixture thereof.

18. The curable mixture of claim 11, wherein the ionic material comprises a compound selected from the group consisting of Ca(OH)₂, Ba(OH)₂, Sr(OH)₂, CaSO₄, BaSO₄, SrSO₄, CaCl₂, BaCl₂, SrCl₂, calcium gluconate, calcium citrate, calcium carbonate, barium gluconate, barium citrate, barium carbonate, strontium gluconate, strontium citrate, strontium carbonate, and a mixture thereof.

19. The curable mixture of claim 18, wherein the ionic material is SrSO₄.

20. The curable mixture of claim 11, wherein the filler material comprises a compound selected from the group consisting of ZnO, a bioactive glass, a non-reactive glass, fumed silica, and a mixture thereof.

21. The curable mixture of claim 20, wherein the filler material comprises microparticles having an average particle size in the range of about 0.1 microns to about 3 microns, wherein the microparticles comprise ZnO, fumed silica or both.

22. (canceled)

23. The curable mixture of claim 11, wherein the X-ray radiopaque material is selected from the group consisting of YbF₃, BaF₂, BaSO₄, SrSO₄, BaWO₄, CaWO₄, SrWO₄ and a mixture thereof.

24-33. (canceled)

34. The curable mixture of claim 11, wherein the ingredients (a), (b), (c), (d) and (e) are selected to provide the curable mixture with a viscosity effective to permit flow into the complex anatomy of a tooth.

35. The curable mixture of claim 34, wherein the tooth comprises a filling space with a diameter in the range of about 150 μm to about 200 μm at the apex.

36. (canceled)

37. The curable mixture of claim 11, wherein the alginate polymer comprises an alginate polymer salt.

38. The curable mixture of claim 37, wherein the alginate polymer salt comprises Na alginate.

39. The curable mixture of claim 11, wherein the alginate polymer comprises a ratio of the guluronic units to the mannuronic units in the range of about 1.5:1 to about 2.3:1.

40. The curable mixture of claim 11, wherein the alginate polymer has a weight average molecular weight in the range of about 75 kDa to about 300 kDa.

41. (canceled)

42. The curable mixture of claim 11, comprising:
8-10 wt. % sodium alginate;
3-5 wt. % CaSO₄;
1-4 wt. % ZnO;
1-3 wt. % fumed silica;
18-30 wt. % YbF₃; and
the balance comprising glycerol, optionally a surface active agent and optionally a retardant.

43. The substantially anhydrous curable mixture of claim 11, comprising:
7-15 wt. % sodium alginate;
3-5 wt. % SrSO₄;
2-8 wt. % ZnO;
1-3 wt. % fumed silica;
18-30 wt. % YbF₃; and
the balance comprising glycerol optionally a surface active agent and optionally a retardant.

44. The substantially anhydrous curable mixture of claim 11, comprising:
7-15 wt. % sodium alginate;
3-5 wt. % CaSO₄;
2-8 wt. % ZnO;
1-3 wt. % fumed silica;
18-30 wt. % YbF₃; and
the balance comprising glycerol optionally a surface active agent and optionally a retardant.

45. A method of filling a tooth, comprising:
identifying a tooth having a cavity in need of filling;
positioning the curable mixture of claim 1 within the cavity; and
curing the curable mixture within the cavity without the need for an external energy source.

46. (canceled)

47. A method of filling a root canal, comprising:
identifying a tooth having a root canal in need of filling;
positioning the curable mixture of claim 11 within the root canal by mixing with a carrier liquid that has a bulk viscosity in the range of about 10 cps to about 200 cps; and
exposing the resultant curable mixture within the root canal to water for a period of time effective to cure the curable mixture.

48. (canceled)

49. (canceled)
